(19) United States
(12) Patent Application Publication
(10) Pub. No.: US 2009/0269310 A1
(43) Pub. Date: Oct. 29, 2009

(54) METHOD FOR OBTAINING HUMAN SMOOTH MUSCULAR CELLS AND USES THEREOF

(75) Inventors: Sophie Le Ricousse, Champigny (FR); Marie-Noelle Lacassagne, Paris (FR); Jean-Pierre Marolleau, Amiens (FR)

Correspondence Address: FOLEY AND LARDNER LLP;SUITE 500, 3000 K STREET NW, WASHINGTON, DC, 20007, US

(73) Assignees: Assistance Publique-Hopitaux De Paris; Institut Des Vaisseaux Et Du Sang

(21) Appl. No.: 12/067,218

(22) PCT Filed: Sep. 19, 2006
(86) PCT No.: PCT/FR06/02144

§ 371 Date: Mar. 18, 2008

(30) Foreign Application Priority Data
Sep. 19, 2005 (FR) 0509557

(51) Int. Cl. A61K 35/34; C12N 5/02; C12N 5/00; C12N 5/08; A61P 35/00; A61P 9/10

(52) U.S. Cl. 424/93.7; 435/387; 435/377; 435/366

(57) ABSTRACT

The invention concerns a method for obtaining in vitro a population of cells comprising essentially human smooth muscular cells expressing calponin and SM-MHC from a sample of human muscular biopsy or from human muscular biopsies differentiated in vitro into skeletal muscle cells. The invention also concerns a composition comprising the isolated smooth muscular cells obtainable by said method as therapeutic principle designed for humans. The invention further concerns the use of the isolated smooth muscular cells for preparing a therapeutic composition designed to replace smooth muscular cells. In particular, the invention concerns the use of said isolated smooth muscular cells for treating ischemia, cancer or any disease requiring revascularization of damaged tissues. Finally, the invention concerns the use of said smooth muscular cells as vector for an active principle for preparing a therapeutic composition designed for humans requiring treatment with said active principle.

This invention concerns a method for obtaining in vitro a population of cells comprising essentially human smooth muscle cells (hSMC) expressing calponin and SM-MHC from a sample of human muscle biopsy or from human muscle biopsies differentiated in vitro into skeletal muscle cells (hSkMC). The invention also concerns a composition comprising the isolated smooth muscle cells obtainable by said method as a therapeutic principle designed for humans. The invention further concerns the use of the isolated smooth muscle cells for preparing a therapeutic composition designed to replace smooth muscle cells. In particular, the invention concerns the use of said isolated smooth muscle cells for treating ischemia, cancer or any disease requiring revascularisation of damaged tissues. Finally, the invention concerns the use of said smooth muscle cells as a vector for an active principle for preparing a therapeutic composition designed for humans requiring treatment with said active principle.

The smooth muscle cells (SMC), present in the vessels, the intestines and the bladder, and the skeletal muscle cells (SkMC) are the two cells types used by the organism to fulfil the function of mechanical contraction. The origin of SMC is complex and depends on their location. In fact during embryogenesis, the SMC precursors can originate from three lines: mesenchymal cells, neural ridge cells or cells derived from the epicard. Recently, the existence of SMC progenitors circulating in peripheral blood has been observed. In fact, different animal models used to study (i) the neointimal formation of vessels, (ii) the outcome of artery grafts or the formation of plaques of atherosclerosis, have made it possible to show that progenitors contained in bone marrow cells participate in these processes, and that they differentiate into SMC.

In adults, the repair of skeletal muscle cells is carried out by the satellite cell population, mononuclear myogenic cells situated under the basal lamina of muscle fibres. But it seems that this cell population is heterogeneous. Moreover, other multipotent cells, isolated from skeletal muscle by flow cytometry using their property of releasing Hoechst dye (1, 2), are capable of differentiating into all blood cells when they are transplanted into mice whose bone marrow has been destroyed by irradiation (2). This cell population is known as the “side population” (SP). It is defined by the expression of the marker Sca1. However, it does not express CD34, ckit and CD45. These cells are able to differentiate into desmin+muscle cells in suitable culture conditions (1). Other studies describe the existence of precursor cells in skeletal muscle with high cell “plasticity” properties (3). Thus skeletal muscle seems to contain several types of stem cells with varied multipotential properties.

The definition of differentiation properties of these stem cells and their control in culture would make it possible to use these easily isolated cells in treatment, notably in repair treatment. These cells, cultivated ex vivo, could then be transplanted, constituting a cell treatment product for autologous treatment of vascular pathologies (post-ischemic revascularisation, atherosclerosis, stabilisation of tumoral vessels, . . . ).

The differentiation of rat SkMS has already been described by Hwang J H. et al. (4) using a method involving a coculture of SkMC with bladder SMC in presence of VEGF, this method making it possible to obtain differentiated SkMC expressing αSMA.

Mention can also be made of the international patent application published with the n^{o} WO 03/027281 (Sakurada Kazuhiro et al.) describing the obtaining of a multipotent stem cell population originating from skeletal muscle interstitial tissue that is able to differentiate into neurones, glial cells, heart muscle cells, adipocytes, vascular endothelial cells, blood cells, bone cells, cartilage cells, pancreas cells and liver cells.

Mention can also be made of the international patent application published with the n^{o} WO 01/94555 (J. P. Marolleau et al.) describing a method for obtaining characterised cell populations of muscular origin and their uses. This document describes in particular a method for obtaining a cell population the dominant cell type of which expresses the CD56 marker and the class I HLA marker, from a muscle tissue biopsy, for the preparation of a cell therapy product for human use, notably by transplant in order to potentiate pharmacological treatments of heart failure.

Therefore it would be desirable to have a method for obtaining in vitro a population of cells essentially comprising smooth muscle cells (SMC), notably from a muscle tissue sample from an individual or patient to be treated using this population.

This is exactly the purpose of this invention.

Thus, in a first aspect, this invention concerns a method for obtaining in vitro a population of cells comprising essentially human smooth muscle cells (hSMC) expressing calponin and smooth muscle myosin heavy chains, known hereafter as SM-MHC, from a sample of human muscle biopsy or from human muscle biopsies differentiated in vitro into skeletal muscle cells (hSkMC),
said human muscle biopsy cells not expressing CD31 and CD14, and, if applicable, lymphocyte markers B and T, and
said hSkMC expressing CD56, desmin and a myogenesis gene selected from the group of genes constituted by the gene MyoD, Myf5 and myogenin, and are able to generate multinuclear myotubes,
characterised in that it comprises the following steps:

A) growing said myoblastic human muscle biopsy cells in a culture medium comprising VEGF (vascular endothelium growth factor), preferably human VEGF, said culture being preferably carried out in the absence of bladder SMC; and

B) recovery of the hSMC obtained in step A).

The term “essentially” as used in the expression “comprising essentially human smooth muscle cells hSMC)” is understood herein to mean notably a population containing at least 50%, preferably at least 60%, 70%, 75% and 80% hSMC with respect to the whole cell population obtained.

In preference, the method according to the invention is characterised in that said hSkMC expressing CD56 and desmin, express the genes MyoD, Myf5 and myogenin.

In preference, the method according to the invention is characterised in that said hSkMC do not express CD34 and CD14.

In preference, the method according to the invention is characterised in that said hSkMC do not express calponin and SM-MHC.

In preference, the method according to the invention is characterised in that said hSMC obtained in step A) express calponin and SM-MHC.

In preference, the method according to the invention is characterised in that said hSMC obtained in step A) do not express the gene MyoD.

In preference, the method according to the invention is characterised in that said hSMC obtained at step A) from human muscle biopsies differentiated in vitro into human skeletal muscular cells (hSkMC), express CD56 and desmin in significantly smaller quantities than said hSkMC used at step A).

In preference, the method according to the invention is characterised in that said hSMC obtained at step A) express Myf5 and myogenin.

In preference, the method according to the invention is characterised in that said culture medium used in step A) further comprises at least one growth factor, preferably human, selected from the group of growth factors consisting of PDGF-BB (platelet derived growth factor, homodimer BB, also called homodimer bb), IGF1(type 1 insulin growth factor), FGFb (basic fibroblast growth factor), HGF (hepatocyte growth factor) and TNFα (alpha tumour necrosis factor), TGFα and all other factors that can have a role in the proliferation or differentiation of SMC.

In preference, the method according to the invention is characterised in that said hSMC are obtained at step A) from human muscle biopsy cells differentiated in vitro into human skeletal muscular cells (hSkMC), characterised in that said hSkMC are obtained from a sample of human muscle biopsy cells by a method comprising the following steps:

a) mincing said muscle biopsy,

b) enzymatic dissociation of the fibres and muscle cells and separation of the individual cells by filtration,

c) putting the muscle cells obtained in this way into culture in a culture reactor of adherent cells in the presence of a growth medium and/or differentiation medium followed, if appropriate, by one or several expansion phases,

d) identification of the cell types present at the different stages of the culture by analysis of the specific cell markers,

e) choosing the culture stage during which the required cell type is a dominant proportion of the cell population,

f) harvesting a population of cells at the culture stage selected in e),

g) if appropriate, deep freezing the cells harvested at step f), notably at the culture stage to be chosen for the preparation of the cell therapy product.

According to a preferred mode of the aforesaid method, the following are carried out:

at step b):
washing the mincings in a medium A followed by enzymatic dissociation of said mincing in the presence of liberase;
separating the individual cells thus obtained by filtering through a sieve followed by centrifugation; and
washing the packed cells thus obtained in a medium B,

at step c):
growing the cells obtained at step b) on a culture plate in a medium C until a degree of confluence of about 20 to 50% is obtained or until the first myotubes appear, then washing the cells in PBS (phosphate buffered saline), FCS (foetal calf serum) then in medium C, where the culture in medium C on enlarged or multi-storey plate units can be carried out again to achieve a degree of confluence of about 90% or the appearance of the first myotubes;
removing the culture medium C and replacing it by a medium D the day before harvesting said cells; and
washing the cells thus obtained in PBS then in medium A,—if appropriate, at the end of step f):
concentrating said cells thus obtained in medium A supplemented with 0.5% (P/V) human albumin serum, and

at step g):
deep freezing said cells thus obtained at step f) is carried out in medium A supplemented with 4% (P/V) human albumin serum and in 7.5% (V/V) DMSO, thawing them at 37° C., then after washing in medium A, suspending them in the culture medium,

and in which steps said media A, B, C and D are media as defined in the international patent application published with the n^{o} WO 01/94555 on Dec. 13, 2001 (pages 24 and 25) namely:

### Medium A:

MCDB 120 medium (Ham et al., 1988) modified: L-valine substituted by D-valine, elimination of phenol red and thymidine.

### Medium B:

Medium A+20% irradiated foetal calf serum+antibiotic.

### Medium C:

Medium B+FGFb (10 ng/ml)+1 μM dexamethasone. Solution D: Phosphate buffered saline (PBS).

In preference, the antibiotic used is gentamycin, notably at 50 μg per ml, or a mixture of penicillin and streptomycin (notably at 100 IU/ml and 100 μg/ml respectively).

In an even more preferred embodiment, the method according to the invention is characterised in that said hSMC are obtained from human muscle biopsy cells previously differentiated into hSkMC obtained according to the method as described in the international patent application published with the n^{o} WO 01/94555, and in which method, the culture stage during which the required hSkMC cell type is a significant proportion of the cell population, is determined by the appearance of a CD56+ phenotype population accounting for at least 50%, preferably at least 60%, 70%, 75% and 80% of the general population.

In preference, said CD56+ phenotype cell population accounting for at least 50%, preferably at least 60%, 70%, 75% and 80% of the general population further possesses at least one of the phenotypes, preferably at least 2, 3 and the 4 phenotypes, selected in the group of phenotypes composed of CD10+, CD13+, desmin+, class 1 HLA and not expressing class 2 HLA.

In a preferred embodiment, the method for obtaining in vitro a population of cells comprising essentially hSMC according to the invention and in which method said hSMC are obtained from a sample of human muscle biopsy cells differentiated in vitro into skeletal muscle cells (hSkMC), is characterised in that at step A), said culture medium comprising VEGF is the MCDB 120 medium as described by Ham et al. (in vitro Cell Dev. Biol., 24, 833-844, 1998) and modified by substitution of the L-valine by D-valine, elimination of phenol red and thymidine.

In a preferred embodiment, the method for obtaining in vitro a population of cells comprising essentially hSMC according to the invention and in which method said hSMC are obtained from a sample of human muscle biopsy cells, is characterised in that at step A), said culture medium comprising VEGF is the M199 medium (such as for example Medium 199 Gibco, Grand Island, N.Y.).

In a preferred embodiment, the method for obtaining in vitro a population of cells comprising essentially hSMC according to the invention is characterised in that at step A), said culture medium comprises 10 ng/ml of VEGF.

In an equally preferred embodiment, the method for obtaining in vitro a population of cells comprising essentially hSMC according to the invention is characterised in that the human muscle biopsy from which said hSMC are obtained directly or previously differentiated into hSkMC, is a biopsy taken from any muscle area, preferably from the leg muscle of the child or adult individual, from whom the sample is taken.

In another aspect, the present invention comprises isolated human smooth muscle cells that can be obtained by the inventive method, said isolated human smooth muscle cells being characterised in that they express calponin and SM-MHC.

In yet another aspect, this invention concerns a composition comprising isolated human smooth muscle cells liable to be obtained or directly obtained from a sample of human muscle biopsy cells or from human muscular biopsy cells differentiated in vitro into skeletal muscle cells by the inventive method, used as a drug.

The present invention also comprises the use of isolated human smooth muscle cells liable to be obtained or directly obtained from a sample of human muscle biopsy or from human muscular biopsies differentiated in vitro into skeletal muscle cells by the inventive method, or the use of the composition as a drug according to the invention for the preparation of a therapeutic composition for human use, notably destined for the individual from whom the muscle biopsy cells cultivated in step A) of said method are taken.

In a preferred embodiment, said therapeutic composition is designed to replace or transplant SMC in humans, preferably autologous replacement or transplant.

In preference, said therapeutic composition is designed for the prevention or treatment of cancers, preferably administered prior to or simultaneously with an anticancerous chemotherapy or radiotherapy treatment.

This is because, contrary to normal vessels, tumoral vessels are structurally and functionally different. The identification of specific markers for tumoral vessels would make it possible to target these vessels without destroying normal vasculature (antiangiogenic therapy) (5). Many studies have shown the functional changes in endothelial cells (EC) of tumoral vessels. And recent results show that the perivascular cells (pericytes or SMC) undergo phenotypic and functional modifications (abnormal shape, expression of new markers, low association with EC, having a cytoplasmic extension that penetrates deeply into the tumoral parenchyma) in the tumoral microenvironment (6-8), thus becoming a new target for antiangiogenic therapies. These physiopathological features of solid tumours compromise the delivery and efficacy of conventional cytotoxic therapies and targeted therapies. A new therapeutic approach would be to make the tumoral vasculature normal before destroying it to facilitate drug delivery (see (9) for a review). In fact, recent results show the efficacy of tumour regression, using the combined therapies, after stabilisation and normalisation of the tumoral vasculature (10). This stabilisation of tumoral vessels could be performed by injecting SMC into or around the tumour site.

This therapeutic approach (injecting isolated human SMC liable to be obtained or directly obtained by the inventive method, with a view to normalising the tumoral vessels) should only be carried out preferably in combination with chemotherapy or radiotherapy. To this end a “therapeutic window” will have to be defined, a period during which the injection of SMC would allow for the greatest effect of the anticancerous treatments.

The vascular “normalisation” will ensure a more functional network, thus enhancing the local diffusion of the drugs, a more homogeneous delivery and the oxygenation of the tumour necessary for certain drugs to operate. This will enable a faster and wider action of the drugs in the tumour, and thus a decrease in the doses administered reducing a priori the severity and frequency of secondary effects. Lastly, the speed and combination of the actions will rapidly limit the proliferation and thus the tumoral resistance phenomena often observed.

The cell therapy proposed here does not constitute a new type of treatment designed to replace current treatments, but will be used as a complement and/or potential synergy to the chemotherapies or radiotherapies currently offered.

In preference, also, said therapeutic composition is designed for the prevention or treatment of ischemia, particularly cardiac or lower limb ischemia.

Many studies carried out in mice and some human protocols have highlighted the improved post-ischemic revascularisation (cardiac or lower limb ischemia) after injecting marrow cells or cells differentiated in vitro. Although at present real integration of these cells into the neovessels seems to be called into question, the basic effects observed are real. Moreover, the role of SMC in these processes could be very important. Recent results show, at the neovascularisation site, the differentiation of marrow cells injected into mice, only into periendothelial cells, and not into endothelial cells (11).

Thus, more particularly, one purpose of the present invention is the use of isolated human smooth muscle cells liable to be obtained or directly obtained from a sample of human muscle biopsy cells or from human muscular biopsy cells differentiated in vitro into skeletal muscle cells by the inventive method, for a composition designed for “normalisation” of the tumoral vasculature or post-ischemic revascularisation.

However, these cells could also be used as a drug designed for a therapeutic use for: atherosclerosis, chronic venous disorders, vascular malformations (such as angiomas).

For this reason, a purpose of the present invention is also the use of isolated human smooth muscle cells liable to be obtained or directly obtained from a sample of human muscle biopsy cells or from human muscular biopsy cells differentiated in vitro into skeletal muscle cells by the inventive method, for the preparation of a therapeutic composition designed for the prevention or treatment of atherosclerosis, arteritis, chronic venous disorders or vascular malformations, particularly angiomas.

Lastly, due to their properties of migrating towards a neoangiogenesis site, these cells can be used as a shuttle or vector for delivering therapeutic active principles such as drugs or anti- or pro-angiogenic factors.

Thus, in another particular aspect, a further purpose of the present invention is the use of isolated human smooth muscle cells liable to be obtained or directly obtained from a sample of human muscle biopsy cells or from human muscular biopsy cells differentiated in vitro into skeletal muscle cells by the inventive method, as a drug, notably as a vector for the administration of a therapeutic active principle or compound, characterised in that:
said isolated human smooth muscle cells are transformed so as to be able to express said active principle or therapeutic compound; or
said isolated human smooth muscle cells have been modified in order to contain said active principle or therapeutic compound that is required to be administered.

The present invention also comprises the use of isolated human smooth muscle cells liable to be obtained or directly obtained from a sample of human muscle biopsy cells or from human muscular biopsy cells differentiated in vitro into skeletal muscle cells by the inventive method, said cells being able to express an active principle or therapeutic compound or containing an active principle or therapeutic compound, for the preparation of a therapeutic composition designed for the prevention or treatment of diseases needing treatment by said active principle or therapeutic compound.

In preference, the use of isolated human smooth muscle cells liable to be obtained or directly obtained from human muscle biopsy cells or from human muscular biopsy cells differentiated in vitro into skeletal muscle cells by the inventive method, for the preparation of a therapeutic composition is characterised in that said composition is administered by an intravenous route or by transplantation.

The captions of the drawings and examples that follow are designed to illustrate the invention without in any way limiting its scope.

### CAPTIONS OF DRAWINGS

FIGS. 1A to 1C. Characterisation of skeletal muscle cells grown in a medium containing FGFb. (FIG. 1A) The flow cytometry analysis shows that these cells express CD56, desmin and CD90 but do not express CD31, CD14 and CD45. In each histogram, the black line corresponds to the cells labelled with a negative control antibody. The broken line corresponds to the cells labelled with the antibody specific to the marker indicated for each histogram. These histograms are representative of 6 samples. (FIG. 1B) RT-PCR analysis. (FIG. 1C) Characterisation of the cultivated skeletal muscle cells by immunocytochemical analysis. The cells are labelled with an anti-IgG control antibody, an anti-αSMA antibody or an anti-SM-MHC followed by labelling with a secondary antibody coupled with peroxidase.

FIGS. 2A to 2C. (FIG. 2A) Morphology of skeletal muscle cells (SkMC) in the medium containing FGFb or VEGF. (FIG. 2B) RT-PCR analysis of the expression of specific skeletal and smooth muscle cell genes in SkMC grown in a medium containing FGFb or VEGF. The cultures were harvested to prepare FRNA at the different times indicated. RT-PCR was carried out and the PCR products were analysed on agarose gels containing ethidium bromide. (FIG. 2C) Detection of the expression of SM-MHC by immunolabelling in the SkMC grown with VEGF for a month. The cells were labelled with either an anti-IgG control antibody or an anti-SM-MHC antibody, followed by labelling with a secondary antibody coupled with peroxidase.

FIGS. 3A to 3C. Photos taken with a phase contrast microscope. The endothelial cells (EC) and the muscle cells are plated together on the surface of a collagen gel. After 24-48 hours, the EC interact with the SMC, originating from the differentiation of the umbilical cord blood precursors (FIG. 3A), or the SMC obtained after growing the skeletal muscle cells (FIG. 3C), to form networks. On the contrary, the SkMC cannot form networks in these conditions (FIG. 3B).

FIGS. 4A to 4C. Matrigel sections, HES labelling. The co-injection of the EC and the SMC obtained from skeletal muscle cells, leads to the formation, in the implant, of vascular lakes (FIG. 4B), with the presence of red blood cells (all the points at the level of the arrows, FIG. 4C at greater magnification). On the contrary, in these same conditions, the SkMC do not form a functional vascular network (FIG. 4A).

FIGS. 5A to 5F. Photos taken with the phase contrast microscope of SkMC (FIGS. 5A to 5D) and SMC (FIGS. 5E and 5F) grown in a medium containing 20% fetal calf serum (FCS) (FIGS. 5A, 5C and 5E) or 2% FCS (FIGS. 5B, 5D and 5F). In order to induce the formation of cells into myotubes, the culture medium of cells reaching 80-90% confluence is changed for a medium supplemented with 2% FCS. The abolition of the formation of myotubes is related to the addition of VEGF to the medium (FIGS. 5C to 5F). The SkMC grown in the presence of VEGF are unable to coalesce into multinuclear myotubes (FIG. 5D).

FIG. 6. The degree of differentiation of the SMC is correlated with the decrease in expression of VEGFR2 and the increase in expression of SRF (Serum response factor). The RT-PCR analysis of 3 different samples of SkMC (1, 2 and 3) grown in presence of FGFb or VEGF. The endothelial progenitor cells (EPC), obtained as described in (16) are used as a positive control for the expression of the VEGF receptors (VEGFR) and negative control for SRF. Whatever the culture conditions, the SkMC and the SMC do not express VEGFR1. In the SkMC, VEGF decreases the expression of VEGFR2, but stimulates the expression of SRF mRNA.

### EXAMPLES

### Methods

### Cell Culture

The SMC differentiated ex vivo from precursors contained in umbilical cord blood were obtained as described above (16). They were grown on type I rat tail collagen (60 μg/ml, SIGMA), in M199 medium (Gibco) supplemented with 20% of 20% foetal calf serum (FCS), 25 mM Hepes buffer (Gibco) and an antibiotic and antifungal solution (Gibco) and recombinant hVEGF at 10 ng/ml (R & D Systems) at 37° C., and in an atmosphere containing 5% CO₂. The culture medium is changed twice a week. The SkMC were grown as previously described (12). In order to induce the differentiation of cells into myotubes, the culture medium of cells at 80-90% confluence was changed for a medium supplemented with 2% FCS, 25 mM Hepes and an antibiotic and antifungal solution (Gibco).

### Immunocytochemistry

The cells were mixed in culture on slides (“chamber slides” Lab-Techn, Poly Labo, Strasburg, France) and fixed with a cold 90% acetone solution. Primary antibodies were used. A murine anti-human αSMA monoclonal antibody (1A4, DAKO) and a murine anti-human smooth muscle myosin heavy chain monoclonal antibody (SMMS-1, DAKO). The (DAKO) EnVision™ System Peroxidase (DAB) kit was used to reveal the αSMA and the SM-MHC. The cells were finally counterstained with hematoxylin.

### Flow Cytometry

An aliquot of cells was directly labelled with antibodies directed against CD31 (5.6E, Coulter), CD45/CD14 (2D1, MφP9, Becton Dickinson), CD56, and CD90. The cells were labelled with an anti-desmin antibody (D33, DAKO) after a permeabilisation step with the permeabilisation reagent Intraprep™ (Coulter). After labelling, the cells were fixed with 1% paraformaldehyde and analysed by flow cytometry (FACStar flow cytometer, Becton Dickinson).

### RT-PCR (Reverse Transcription-Polymerase Chain Reaction)

The total RNA was extracted with RNAXEL® (EUROBIO, Les Ulis, France) according to the supplier's instructions. The cDNA synthesis was carried out using the “1^{st} strand cDNA synthesis kit for RT-PCR (AMV)” (Boerhinger Mannheim). Thus, the cDNA fragment of interest was able to be amplified by PCR. The PCR mixture contained 1× reaction buffer, 1.5 mM MgCl₂, 0.2 mM deoxynucleotide mixture, 0.5 units of Taq polymerase and 0.2 μM of sense and antisense primers. The following primers were used for the RT-PCR: GAPDH sense (SEQ ID NO: 1): 5′-CCA TGG AGA AGG CTG GGG-3′, antisense (SEQ ID NO: 2): 5′-CAA AGT TGT CAT GGA TGA CC-3′, calponin sense (SEQ ID NO: 3): 5′-AGA-AGT-ATG-ACC-ACC-AGC-3′, antisense (SEQ ID NO: 4): 5′-TAG-AGC-CCA-ATG-ATG-TTC-CG-3′, SM22α sense (SEQ ID NO: 5): 5′-GCA-GTC-CAA-AAT-TGA-GAA- GA-3′, antisense (SEQ ID NO: 6): 5′-CTG-TTG-CTG-CCC-ATT-TGA-AG-3′, Myogenin sense (SEQ ID NO: 7): 5′-AGC-GCC-CCC-TCG-TGT-ATG-3′, antisense (SEQ ID NO: 8): 5′-TGT-CCC-CGG-CAA-CTT-CAG-C-3′, MyoD sense (SEQ ID NO: 9): 5′-CGG-CGG-CGG-AAC-TGC-TAC-GAA-3′, antisense (SEQ ID NO: 10): 5′-GGG-GCG-GGG-GCG-GAA-ACT-T-3′, Myf5 sense (SEQ ID NO: 11): 5′-ACC- ATG-GAT-CGG-CGG-AAG-G-3′, antisense (SEQ ID NO: 12): 5′-AAT-CGG-TGC- TGC-CAA-CTG-GAG-3′, VEGF-R1 sense (SEQ ID NO: 13): 5′-CGA CCT TGG TTG TGG CTG ACT-3′, antisense (SEQ ID NO: 14): 5′-CCC TTC TGG TTG GTG GCT TTG-3′, VEGF-R2 sense (SEQ ID NO: 15): 5′-AAC AAA GTC GGG AGA GGA-3′, antisense (SEQ ID NO: 16): 5′-TGA CAA GAA GTA GCC AGA AGA-3′, SRF sense (SEQ ID NO: 17): 5′-AGT-GTG-TGG-GGG-AGA-TTC-TG-3′ and antisense (SEQ ID NO: 18): 5′-TCT-CCC-TAG-CAA-CAG-CCC-TA-3′.

### Example 1

### Culture Conditions Making it Possible to Obtain Large Quantities of Human SMC from Progenitor Cells or Differentiated Skeletal Muscle Cells

### A) Initial Cell Population:

The method of the invention relates to a method for obtaining a cell population in which one dominant cell type is the smooth muscle cell type. This method can be applied either directly to muscle biopsy cells, or after an initial phase of differentiation of biopsy cells into SkMC and amplification of these cells. The conditions for obtaining muscle biopsies and SkMC from these biopsies and their phenotypic characterisation are defined in the international patent application published with the n^{o} WO 01/94555 (J. P. Marolleau et coll.). Moreover, the biopsy cells do not express CD31 and CD14.

Starting from a few grams of muscle biopsy, it is possible to obtain several hundred million SkMC. These cells express CD56, desmin and myogenesis genes such as Myf5 and myogenin. However, they do not express CD34, CD14 and specific markers of SMC such as: calponin and SM-MHC. These cells are able to coalesce and give rise to multinuclear myotubes.

B) Differentiation of Skeletal Muscle Cells into Smooth Muscle Cells:

The cells from the biopsy, or after differentiation into SkMC, are plated in MCDB or M199 medium in the presence of VEGF alone or with other growth factors (PDGF-BB, IGF1, FGFb, HGF or TNFα).

### Solutions and Media Used:

### Medium A:

Modified MCDB 120 medium (Ham et al., 1988): L-valine substituted by D-valine, removal of phenol red and thymidine.

### Medium B:

Medium A+20% irradiated foetal calf serum+antibiotic (gentamycin at 50 μg per ml, or 100 IU/ml for penicillin or 100 μg/ml for streptomycin).

### Medium C:

Medium B+FGFb (10 ng/ml)+1 μM dexamethasone. Solution D: Phosphate buffered saline (PBS) (see the international patent application published with the n^{o} WO 01/94555 pages 24 et 25)

### Medium E: M199

Medium F: M199+20% decomplemented foetal calf serum+Hepes (25 mM)+antibiotic (penicillin, streptomycin) and, if necessary, an antimycotic (such as fungizone at 25 μg/ml, or as indicated above).

Medium G: Medium F (M199+FCV+Hepes+antibiotic)+VEGF (10 ng/ml).

Medium H: Medium B (MCDB+FCV+antibiotic+dexamethasone)+VEGF (10 ng/ml).

The media containing the different growth factors described above.

The expression of genes associated with the differentiation into SkMC or SMC, during culture, was analysed by polymerase chain reaction after reverse transcription (RT-PCR) flow cytometry and immunocytochemistry. Thus, after a month of culture in medium M199 or MCDB 120 containing VEGF, these cells express the messenger RNA and proteins specific to SMC. So they express calponin and SM-MHC. In parallel, they express desmin and CD56 much less strongly and no longer express MyoD at all. However, the expression of transcription factors Myf5 and myogenin persists.

The inventors have also shown that these phenotype modifications lead to different functional properties.

### Example 2

### Differentiation of Skeletal Muscle Cells to Smooth Muscle Cells

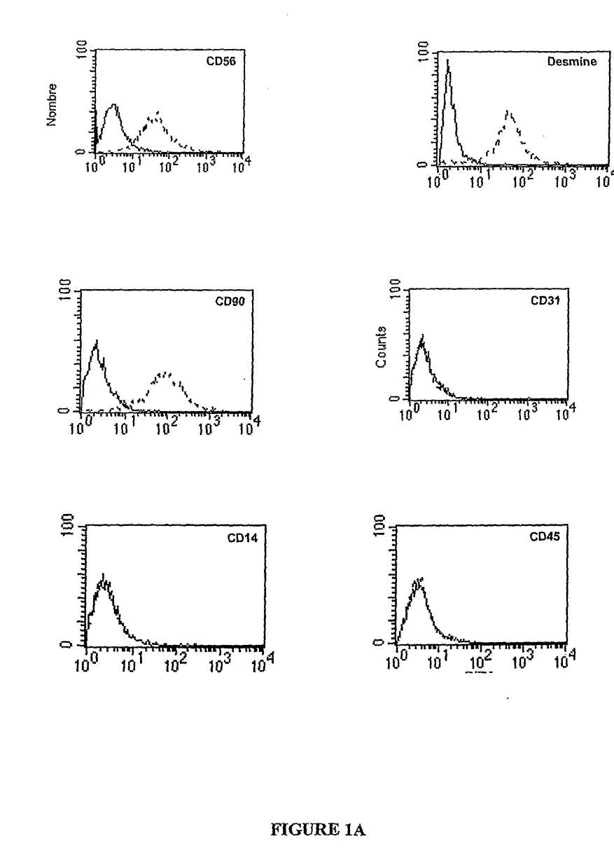

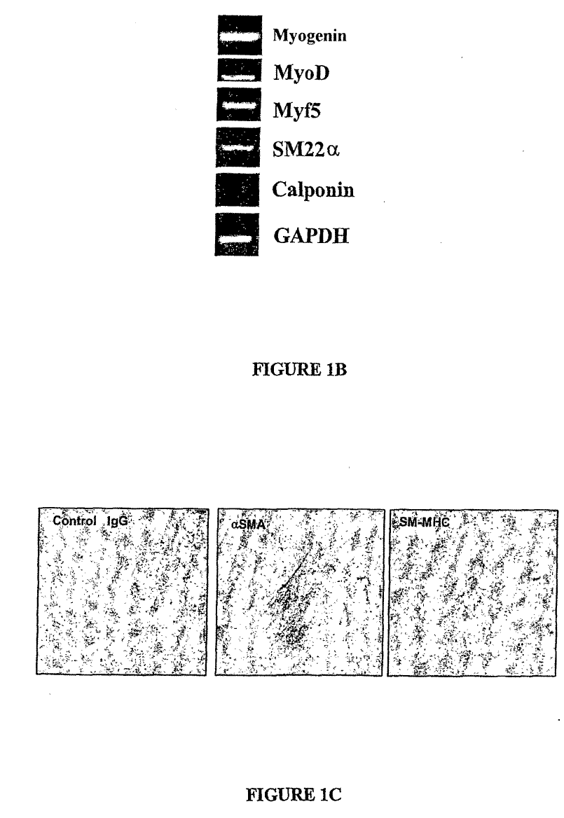

Muscle biopsy cells were first put in culture for expansion in a medium containing FGFb as described above (12). To characterise the phenotype of these cells, analyses using flow cytometry (FACS), reverse transcription polymerase chain reaction (RT-PCR) and immunocytochemistry were carried out. The FACS analysis has shown that most of these cells are positive for CD56 (80.30+19.50%), desmin (92.30+8.48%) and CD90 (91.32+10.19%) and negative for the endothelial marker CD31, the monocyte marker CD14, and the leukocyte marker CD45 (FIG. 1A). The RT-PCR analysis has shown that the cells express markers related to myogenic cells such as Myf5, MyoD and Myogenin (FIG. 1B). The cells also express the specific smooth muscle cell markers SM22α (FIG. 1B) and αSMA (FIG. 1C). But certain isoforms of smooth muscle cells have been detected in developing or regenerating skeletal muscle cells (14, 15. These cells do not express markers of differentiated smooth muscle cells such as calponin (FIG. 1B) and SM-MHC (FIG. 1C).

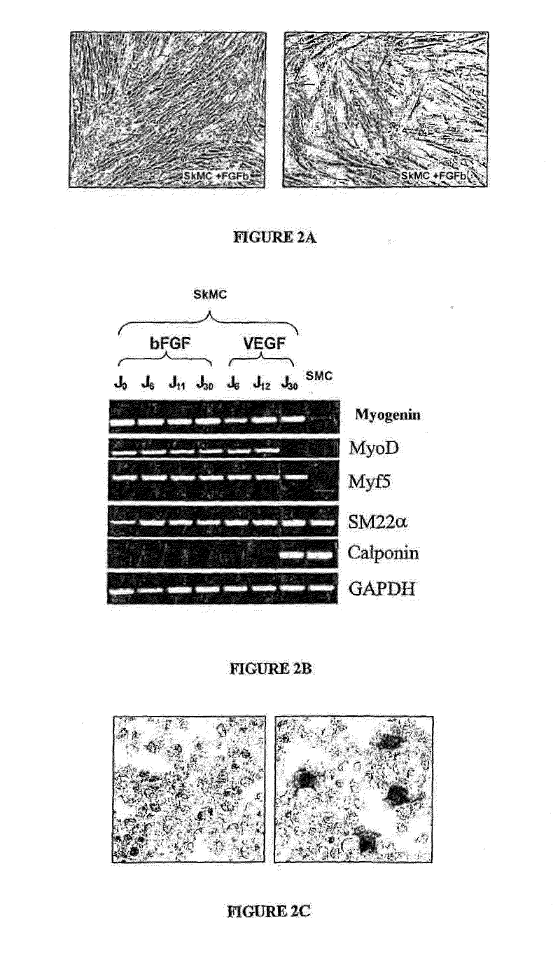

The cells were then put in culture in a medium containing VEGF (10 ng/ml). After 7 days, changes in cell morphology were observed (FIG. 2A). The RT-PCR technique was used to compare the changes of expression of genes during culture between skeletal muscle and smooth muscle cells. This analysis was carried out on the RNA obtained from cells on days 0, 6, 11 or 12 and 30 after putting in culture, and the results are given in FIG. 2B. It was observed that genes coding for SM22α, Myogenin and Myf5 were expressed at a similar level, whatever the culture conditions and throughout the whole duration of the culture. The skeletal muscle cells (SkMC) put in culture with FGFb show no calponin expression. But after a month of culture with VEGF, these cells express calponin mFRNA and concurrently no longer express MyoD (FIG. 2B). The expression of SM-MHC confirms that these cells have adopted a smooth muscle cell phenotype (FIG. 2C). Due to the variability in structural gene expression in the smooth muscle cells (SMC), received opinion is that in order for a cell to be characterised as a differentiated SMC, the expression of several isoforms of structural genes associated with smooth muscle needs to be demonstrated. Thus, the expression of SM22α, calponin and SM-MHC in the SkMC is a strong indication that certain cells in the culture have adopted a differentiated SMC identity. So as to define the best culture conditions, other growth factors known to induce the differentiation or proliferation of SMC were tested. Thus culture conditions in which PDGF BB and/or FGFb and/or HGF and/or TGFβ and/or IGF1 were added to the VEGF were tested. But no significant effect on the differentiation of SkMC into SMC or on their proliferation was observed.

Frid M. G. et al. (13) have shown that mature bovine endothelium contains cells which, in vitro, can acquire a SMC phenotype by a transdifferentiation process. It has been confirmed here by FACS analysis that the cells in culture are not contaminated by endothelial cells (EC). They do not express markers related to endothelial cells such as CD31 (FIG. 1A). Further, the hypothesis can be posited that the observed phenomenon is not a simple contamination by SMC from an external source. This is because all the biopsies tested, which show the expression of genes related to skeletal muscle myogenin, MyoD, Myf5 and desmin, undergo differentiation into smooth muscle.

### Example 3

### Functional Tests

The acquisition of smooth muscle cell markers does not necessarily mean that these cells are able to differentiate into mature SMC.

### A) Culture in Type I Collagen Gel (3D Culture)

### Protocol:

Culture in type I collagen gel (3D culture) (BD Biosciences, Bedford, Mass.) was carried out according to the supplier's recommendations, namely:—0.5 ml of 1 mg/ml type I rat tail collagen (Becton Dickinson) was poured into 35 mm diameter culture dishes (Nunc, Fisher Scientific, Elancourt, France) and left to polymerise for 1 hour at 37° C. A total of 400 000 cells (200 000 of each type of cell when endothelial cells are mixed with muscle cells) are then plated on the gel surface and put in culture for 24 hours under the different culture conditions. The formation of vascular networks was then observed with a phase contrast microscope and a “charge-coupled” videocamera Kappa CF1 IDSP.

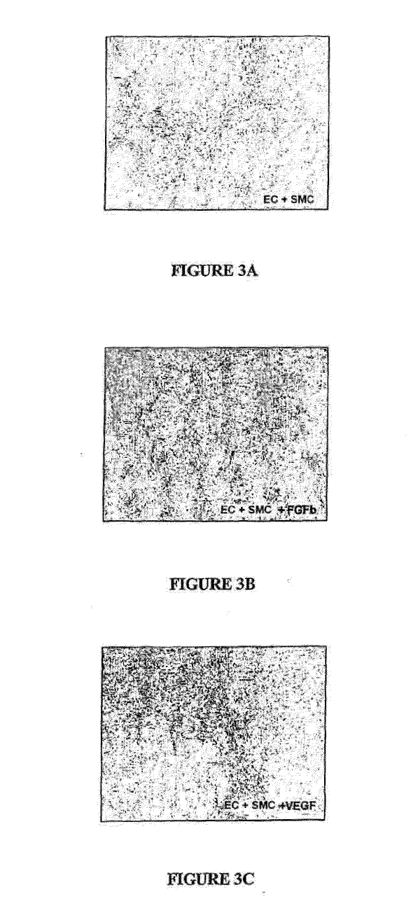

### Results (see FIGS. 3A to 3C):

The ability of the cells to organise themselves in a 3D collagen structure was then analysed. It was shown that the endothelial cells (EC) and the SMC interacted with each other to form capillary type networks in vitro in 3D culture. The ability of SkMC grown with FGFb, and SkMC grown with VEGF to associate with EC and form capillary type networks in vitro was compared. It was observed that SkMC grown with VEGF are able to interact with EC forming compact tubular networks (FIG. 3C), whereas SkMC grown with FGFb do not interact with the EC in the same conditions and form no vessel networks (see FIG. 3B).

### B) Matrigel Model

The ability of each cell type, SkMC with FGFb or with VEGF, to form tubular vessel structures in a Matrigel implant model in NOD-SCID immunosuppressed mice was tested. Protocol:

D0: A 0.2 ml Matrigel implant (BD Biosciences) (containing 0.5 mg/ml of FGFb) was injected subcutaneously in the backs of NOD-SCID immunosuppressed mice.

D1: In the morning, the mice were sub-lethally irradiated (325 rad). In the afternoon, 500 000 cells were injected intravenously via the caudal vein.

D10: The animals were sacrificed, the implant was recovered and embedded in paraffin. An HES stain (hemalin, eosin, safranin) was made and examined (enlargement 4×, 40×).

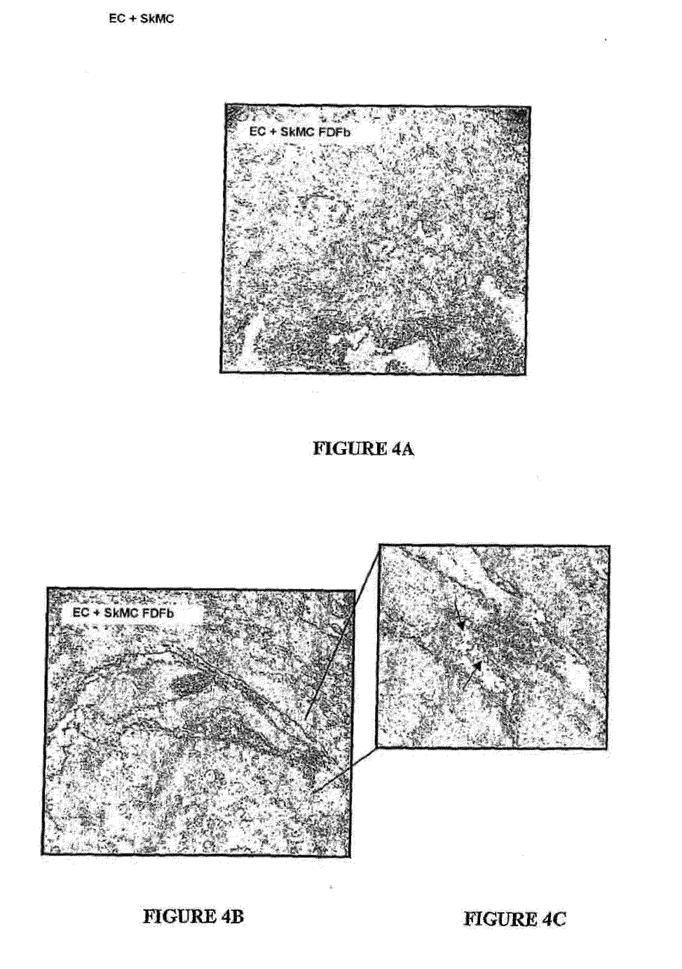

### Results (see FIGS. 4A to 4C):

These results were obtained and reproduced for SkMC originating from 3 different patients or from biopsies of 5 different patients.

In the Matrigel implant, the administration of EC and SMC or EC and SkMC grown with VEGF leads to the formation of many tubular type structures and the presence of erythrocytes is shown up under light, demonstrating the existence of a functional vascular structure (FIGS. 4B and 4C). Conversely, the administration of EC and SkMC grown with FGFb does not lead to the formation of any tubular type structure and causes the formation of disorganised cell aggregates (FIG. 4A).

### C) The SkMC Grown in Presence of VEGF No Longer Coalesce in Multinuclear Myotubes.

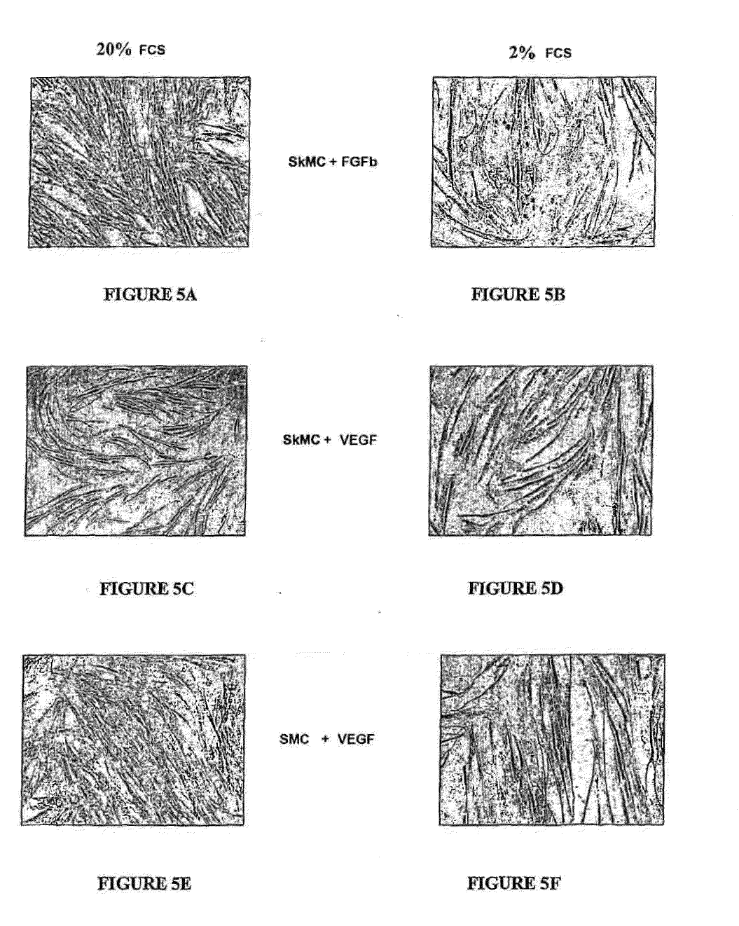

(See FIGS. 5a to 5f)

The coalescence of individual myoblasts into multinuclear myotubes constitutes the terminal differentiation of SkMC. The formation of myotubes was examined by putting SkMC in culture, with FGFb or VEGF, at the same initial density, and then changing the culture conditions for a medium with 2% foetal calf serum. In these conditions, myotubes appeared 10 days after putting into culture. Contrary to SkMC grown with FGFb (FIG. 5B), the SkMC grown with VEGF (FIG. 5D), like the SMC (FIG. 5F), are incapable of coalescing into multinuclear myotubes. So these cells have lost the ability to form multinuclear myotubes.

### D) The VEGF is Involved in Inducing the Transition of the SkMC Phenotype to the SMC Phenotype by Increasing Expression of Serum Response Factor SRF.

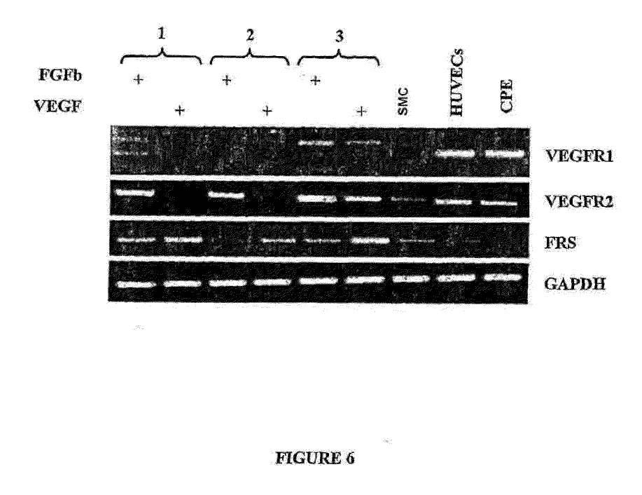

The VEGF is a major regulator of the formation of blood vessels during body development and in adults. In order to explore the possible signal transduction path for VEGF, the expression of VEGFR1 and VEGFR2 receptors was analysed in SkMC and SMC. RT-PCR analysis showed that SkMC express a large quantity of VEGFR2. But when these cells are grown in a medium containing VEGF a decrease in the expression of VEGFR2 is observed (FIG. 6). And, whatever the culture conditions, we have not shown the detection of VEGFR1 expression. Therefore these results suggest the role of VEGFR2 in the mediation of the transdifferentiation of SkMC into SMC stimulated by VEGF. SRF is a key regulator of many cellular early response genes that are known to be involved in cell growth and differentiation. Some results suggest that one or several cofactors of SRF restricted to the SkMC or SMC line could function together with SRF to activate the transcription of line-specific genes. In order to understand mechanisms participating in the differentiation of SkMC into SMC, the expression of SRF was compared in the cells before and after adding VEGF. It was observed that when the SkMC are grown in a medium containing VEGF the expression of SRF mRNA was increased (FIG. 6).

### REFERENCES

1: Gussoni E. et al.: Dystrophin expression in the mdx mouse restored by stem cell transplantation. Nature 1999, 401:390-394.
2: Jackson K A. et al. From the cover: hematopoietic potential of stem cells isolated from murine skeletal muscle. PNAS 1999, 96: 14482-14486.
3: Tamaki T. et al.: Identification of myogenic-endothelial progenitor cells in the interstitial spaces of skeletal muscle. J Cell Biol 2002; 157:571-577.
4: Hwang J H. et al.: Differentiation of stem cells isolated from rat smooth muscle. Mol Cells 2004; 17(1):57-61.
5: Benjamin L. E. et al.: Selective ablation of immature blood vessels in established human tumors follows vascular endothelial growth factor withdrawal. J Clin Invest 1999; 103:159-165.
6: Morikawa S. et al.: Abnormalities in pericytes on blood vessels and endothelial sprouts in tumors. Am J Pathol 2002; 160:985-1000.
7: Berger M. et al.: Regulator of G protein signaling-5 induction in pericytes coincides with active vessel remodeling during neovascualrization. Blood 2004 epub Oct. 30, 2004.
8: Bergers G. et al.: benefits of targeting both pericytes and endothelial cells in the tumor vasculature with kinase inhibitors. J Clin Invest 2003; 111: 1287-1295.
9: Jain R. K.: Normalizing of tumor vasculature: an emerging concept in antiangiogenic therapy. Science 2005; 307:58-62.
10: Ganss R. et al.: Combination of T-Cell therapy and trigger of inflammation induces remodeling of the vasculature and tumor eradication. Cancer Res 2002; 62:1462-1470.
11: Rajantie I. et al.: Adult bone marrow-derived cells recruited during angiogenesis comprise precursors for periendothelial vascular mural cells. Blood 2004; 104:2084-2086.
12: Menaschè P, Hagège A A, Vilquin J T, et al. Autologous skeletal myoblast transplantation for severe postinfarction left ventricular dysfunction. J. Am. Coll. Cardiol. 41, 1078-1083 (2003).
13: Frid M G, KaIe V A, Stenmark K R. Mature vascular endothelium can give rise to smooth muscle cells via endothelium-mesenchymal transdifferentiation. In vitro analysis. Cire. Res. 90, 1189-1196 (2002).
14: Li L, Miano J M, Cserjesi P, Oison E N. SM22 alpha, a marker of adult smooth muscle, is expressed in multiple myogenic lineages during embryogenesis. Cire. Res. 78, 188-195 (1996).
15: Woodcock-Mitchell J, Mitchell J J, Low R B, Kieny M, Sengel P, Rubbia L, Skalli O, Jackson B, Gabbiani B. α-Smooth muscle actin is transiently expressed in embryonic rat cardiac and skeletal muscles. Differentiation 39, 151-166 (1998). 16: Le Ricousse-Roussanne S, Barateau V, Contreres J O, et al. Ex vivo differentiated endothelial and smooth muscle cells from human cord blood progenitors home to the angiogenic tumor vasculature. Cardiovas. Res. 62, 176-184 (2004).

1. A method for obtaining in vitro a population of cells comprising essentially human smooth muscle cells (hSMC) expressing calponin and SM-MHC from a sample of human muscle biopsy cells or from human muscle biopsy cells differentiated in vitro into skeletal muscle cells (hSkMC).
said human muscle biopsy cells not expressing CD31 and CD14, and, if applicable, lymphocyte markers B and T, and
said hSkMC expressing CD56, desmin and a myogenesis gene selected from the group of genes constituted by the genes MyoD, Myf5 and myogenin, and able to generate multinuclear myotubes,
comprising the following steps:
A) growing said myoblastic human muscle biopsy cells in a culture medium comprising VEGF, said culture being carried out in the absence of bladder SMC; and
B) recovery of the hSMC obtained in step A).

2. The method according to claim 1, wherein said hSkMC expressing CD56 and desmin, express the genes MyoD, Myf5 and myogenin.

3. The method according to claim 1, wherein said hSkMC do not express CD34 and CD14.

4. The method according to claim 1, wherein said hSkMC do not express calponin and SM-MHC.

5. The method according to claim 1, wherein said hSMC obtained at step A) express calponin and SM-MHC.

6. The method according to claim 1, wherein said hSMC obtained at step A) do not express the gene MyoD.

7. The method according to claim 1, wherein said hSMC obtained at step A) from human muscle biopsy cells differentiated in vitro into human skeletal muscle cells (hSkMC), express CD56 and desmin in significantly smaller quantities than said hSkMC used at step A).

8. The method according to claim 7, wherein said hSMC obtained at step A) express Myf5 and myogenin.

9. The method according to claim 1, wherein said culture medium used at step A) further comprises at least one growth factor selected from the group of growth factors consisting of PDGF-BB, IGF1, FGFb, HGF and TNFα, TGFβ and all other factors that can have a role in the proliferation or differentiation of SMC.

10. The method according to claim 1, wherein said hSMC are obtained from a sample of human muscle biopsy cells differentiated in vitro into human skeletal muscular cells (hSkMC), wherein said hSkMC are obtained from a sample of human muscle biopsy cells by a method comprising the following steps:
a) mincing said muscle biopsy,
b) enzymatic dissociation of the fibres and muscle cells and separation of the individual cells by filtration,
c) putting the muscle cells obtained in this way into culture in a culture reactor of adherent cells in the presence of a growth medium and/or differentiation medium followed if appropriate by one or several expansion phases,
d) identification of the cell types present at the different stages of the culture by analysis of specific cell markers,
e) choosing the stage of culture during which the required cell type is a dominant proportion of the cell population,
f) harvesting a population of cells at the culture stage selected in e), and
g) if appropriate, deep freezing the cells harvested in step f).

11. The method according to claim 10, wherein:
at step b), the following steps are carried out:
washing the mincings in a medium A followed by enzymatic dissociation of said mincing in the presence of liberase;
separating the individual cells thus obtained by filtering through a sieve followed by centrifugation; and
washing the packed cells thus obtained in a medium B;
at step c), the following steps are carried out:
growing the cells obtained at step b) on a culture plate in a medium C until they are 20 to 50% confluent or until the first myotubes appear, then washing the cells in PBS, FCS then in medium C, and then they can be cultured again in medium C on large plates or in culture flasks to achieve a degree of confluence of about 90% or the appearance of the first myotubes,
removing the culture medium C and replacing it by medium D the day before harvesting said cells,
washing the cells thus obtained in PBS then in medium A; and,
if necessary, concentrating said cells thus obtained at the end of step f) in medium A supplemented with 0.5% (P/V) human albumin serum;
at step g) deep freezing said cells thus obtained at step f) is carried out in medium A supplemented with 4% (P/V) human albumin serum and in 7.5% (V/V) DMSO, thawing them at 37° C., then after washing in medium A, suspending them in the culture medium, and in which steps said media A, B, C, and E are the following:
Medium A:
Modified MCDB 120 medium (Ham et al., 1988): L-valine substituted by D-valine, removal of phenol red and thymidine,
Medium B:
Medium A+20% irradiated foetal calf serum+antibiotic,
Medium C:
Medium B+FGFb (10 ng/ml)+1 μM dexamethasone, Solution or medium D: Phosphate buffered saline (PBS).

12. The method according to claim 10, wherein the culture stage during which the required hSkMC cell type is a significant proportion of the cell population, is determined by the appearance of a CD56+ phenotype population accounting for at least 50% of the general population.

13. The method according to claim 12, wherein said CD56+ phenotype population accounting for at least 50% of the general population further possesses at least one of the phenotypes, preferably at least 2, 3 and the 4 phenotypes, selected in the group of phenotypes composed of CD10+, CD13+, desmin+, class 1 HLA.

14. The method according to claim 1, wherein said hSMC are obtained from a sample of human muscle biopsies differentiated in vitro into human skeletal muscular cells (hSkMC), wherein at step A), said culture medium comprising VEGF is MCDB 120 medium modified by substitution of L-valine by D-valine, removal of phenol red and thymidine.

15. The method according to claim 1, wherein said hSMC are obtained from a sample of human muscle biopsies characterised in that at step A), said culture medium comprising VEGF is M199 medium.

16. The method according to claim 1, wherein at step A), said culture medium comprises 10 ng/ml of VEGF.

17. The method according to claim 1, wherein the human muscle biopsy from which said hSMC are obtained directly or previously predifferentiated into hSkMC, is a biopsy taken from any muscle territory of the individual from whom the sample is taken.

18. The method according to claim 1, wherein the human muscle biopsy from which said hSMC are obtained directly or previously predifferentiated into hSkMC, is a biopsy taken from the leg muscle territory of the individual from whom the sample is taken.

19. The method according to claim 1, wherein the human muscle biopsy from which said hSMC are obtained directly or previously predifferentiated into hSkMC, is a biopsy taken from the muscle territory of a child or adult individual.

20. Isolated human smooth muscle cells able to be obtained by the method according to claim 1, wherein they express calponin and SM-MHC.

21. A therapeutic composition comprising isolated human smooth muscle cells able to be obtained by the method according to claim 1, that express calponin and SM-MCH, or obtained by the method according to claim 1, and a culture medium.

22-29. (canceled)

30. A method for SMC replacement in humans, comprising administering to a human in need thereof a therapeutical effective amount of the composition according to claim 21.

31. The method of claim 30, wherein the administering is intravenous or by transplantation.

32. A method for the prevention or treatment of atherosclerosis, arteritis, chronic venous disorders, vascular malformations, particularly angiomas in humans, comprising administering to a human in need thereof a therapeutically effective amount of the composition according to claim 21.

33. A method of treating cancer, comprising administering to a human in need thereof a therapeutical effective amount of the composition according to claim 21.

34. A method for the prevention or treatment of cancers administered prior to or concurrently with an anticancer treatment by chemotherapy or radiotherapy comprising administering to a human in need thereof a composition according to claim 21.

35. A method for the prevention or treatment of ischemia, particularly of the heart or lower limbs administering to a human in need thereof a composition according to claim 21.

36. An isolated human smooth muscle cell comprising or able to express an active principle.
